(19) United States
(12) Patent Application Publication
(10) Pub. No.: US 2008/0199441 A1
(43) Pub. Date: Aug. 21, 2008

(54) Use of ex-vivo cultured hematopoietic cells for treatment of peripheral vascular diseases

(76) Inventor: Tony Peled, Mevasseret Zion (IL)

Correspondence Address: MINTZ, LEVIN, COHN, FERRIS, GLOVSKY AND POPEO, P.C;ATTN: PATENT INTAKE CUSTOMER NO. 30623, ONE FINANCIAL CENTER, BOSTON, MA, 02111, US

(21) Appl. No.: 11/983,609

(22) Filed: Nov. 8, 2007

Related U.S. Application Data
(60) Provisional application No. 60/857,787, filed on Nov. 9, 2006.

(51) Int. Cl. A61K 45/00; C12N 5/02; A61P 9/14

(52) U.S. Cl. 424/93.7; 424/93.73; 435/384

(57) ABSTRACT

Methods for cell therapy of peripheral vascular disease by local administration of ex-vivo cultured hematopoietic cells are provided.

### RELATED APPLICATION

This application claims the benefit of priority from U.S. Provisional Patent Application No. 60/857,787, filed Nov. 9, 2006, the contents of which is incorporated by reference as if fully set forth herein.

### FIELD AND BACKGROUND OF THE INVENTION

The present invention, in some embodiments thereof, relates to cell therapy of peripheral vascular disease and, more particularly, but not exclusively, to ex-vivo cultured hematopoietic cells and their use for the treatment of peripheral vascular disease.

Peripheral artery occlusive disease (PAOD, also known as peripheral vascular disease or PVD) is a collator for all diseases caused by the obstruction of large peripheral arteries, which can result from atherosclerosis, inflammatory processes leading to stenosis, an embolism or thrombus formation. It causes either acute or chronic ischemia. PVD refers to diseases of blood vessels outside the heart and brain, and is often a narrowing of vessels that carry blood to the legs, arms, stomach or kidneys. An estimated more than 10 million Americans are affected by circulatory problems (generally in the legs) associated with peripheral artery disease (PAD). It is caused by arteriosclerosis, the obstruction and hardening of arteries that can lead to heart attacks. Although about half of those with PAD experience few or no symptoms, others report varying levels of pain and other symptoms including paresthesia (numbness) and ulceration of the legs and feet. Early treatment is similar to life-style changes directed to reducing risk of heart disease, such as diet, smoking cessation, weight loss and if appropriate, cholesterol lowering drugs. If PAD progresses, patients may require an artery bypass graft or angioplasty procedure for widening the occluded blood vessel. However as many as 12 percent of PAD patients are not surgical candidates, and as many as 30,000 to 50,000 people per year in the United States are required to undergo amputation due to PAD. For many of these severely affected PAD patients, their quality of life is irreversibly compromised.

Currently, PVD is treated conservatively and the first-line therapeutic options include pain medication (palliative), “anti-thrombotic” drugs and therapy. However, in the case of severe and/or progressive symptoms, surgical interventions such as angioplasty, bypass surgery, atherectomy and even amputation may be necessary. Despite major advances in these areas, PVD remains a clinical challenge, with often unsatisfactory results in treated patients with advanced stage disease. Thus, there is an increasingly great clinical demand for novel treatment options aimed at stimulating collateral blood vessel formation, increasing vascularity and improving skeletal muscle function.

Induction of therapeutic angiogenesis by stem cell implantation could provide therapeutic benefit and may help in wound healing and limb salvage in patients with PVD. Angiogenesis (collateral vessel formation) can be achieved either by the administration of growth factors or expression of genes encoding the growth factor proteins. Recent studies have suggested that marrow and blood hematopoietic stem cells may contribute to nonhematopoietic tissue repair in multiple organ systems. In animal models and more recently in limited human trials, hematopoietic stem cells have been reported to contribute to neoangiogenesis (Bone Marrow Transplant. 2003 Aug; 32 Suppl 1:S29-31; Asahara et al. Gene Therapy, 2000;9:451-57; Yla Herttuala et al. Nature Medicine, 2003;9:694-701; Tateishi et al, Lancet 2002 360:427-35; Higashi et al, Circulation 2004;109:1215-1218; for a review, see Perin et al. Circulation 2003 107:935).

Expansion of Hematopoietic Cell Populations:

While many methods for stimulating proliferation of hematopoietic cell populations have been disclosed [see, for example, Czyz et al, Biol Chem 2003; 384:1391-409; Kraus et al. (U.S. Pat. No. 6,338,942, issued Jan. 15, 2002); Rodgers et al. (U.S. Pat. No. 6,335,195 issued Jan. 1, 2002); Emerson et al. (Emerson et al., U.S. Pat. No. 6,326,198, issued Dec. 4, 2001) and Hu et al. (WO 00/73421 published Dec. 7, 2000) and Hariri et al (US Patent Application No. 20030235909)] few provide for reliable, long-term expansion, without the accompanying differentiation that naturally occurs with growth of hematopoietic cells in culture.

Hematopoietic Cellular Differentiation

A single HSC can give rise to all types of hematopoietic cells, and is found in very low numbers predominantly in the bone marrow (although HSCs are also found in umbilical cord blood (UBC) and other tissues). Studies characterize human HSCs as small quiescent cells that express high levels of the surface glycoprotein CD34 (CD34+), and low or undetected levels of markers such as CD33, CD38, thy-1, and CD71, which designate a more mature progenitor population. CD34+CD38− cells (which represent <10% of the limited CD34+ cell population) can give rise to both lymphoid and myeloid cells in vitro, repopulate immune-compromised mice to high degrees, and appear to be critical to hematopoietic recovery of patients receiving autologous blood cell transplantation. In all the currently used methods of ex-vivo expansion, significant accumulation of intermediate and late progenitors is achieved, with limited expansion of the CD34+CD38− subpopulation, a significant obstacle to any prospect of utilizing cultured early hematopoietic cells in cell and gene therapy.

Until recently, limited expansion of progenitor cell subsets has been achieved either by growing the stem cells over a feeder layer of fibroblast cells, or by growing the cells in the presence of the early acting cytokines thrombopoietin (TPO), interleukin-6 (IL-6), an FLT-3 ligand and stem cell factor (SCF) (Madlambayan G J et al. (2001) J Hematother Stem Cell Res 10: 481; Punzel M et al. (1999) Leukemia 13: 92; and Lange W et al. (1996) Leukemia 10: 943). Recently, however, other methods for expansion of hematopoietic cells ex-vivo have been disclosed. PCT IL99/00444 of Peled et al., filed Aug. 17, 1999, which is incorporated by reference as if fully set forth by reference herein, disclosed methods of restricting differentiation of ex vivo cultured hematopoietic cells by treating the cells with chelators of transitional metals. While reducing the invention to practice, Peled et al uncovered that heavy metal chelators having a high affinity for copper, such as tetraethylpentamine (TEPA), greatly enhanced the fraction of CD34⁺ cell and their long-term clonability in cord-blood-derived, bone marrow-derived, and peripheral blood derived hematopoietic cells, grown without a feeder layer. Facilitation of proliferation while inhibiting differentiation was also observed in erythroid progenitor cells, cultured mouse erythroleukemia cells, embryonal stem cells, and hepatocytes in primary hepatocyte culture treated with TEPA.

PCT/IL03/00062, also to Peled et al., filed Jan. 23, 2003, which is incorporated by reference as if fully set forth herein, discloses a similar effective promotion of long term ex vivo hematopoietic cell proliferation, while inhibiting differentiation, using TEPA-Cu chelates as well as the chelator TEPA. Surprisingly, this effect of TEPA and TEPA-chelates was also demonstrated using as a starting population an un-selected peripheral mononuclear fraction. The results described there-in clearly show that hematopoietic cells may be substantially expanded ex vivo, continuously over at least a 12 week period, in a culture of mixed (mononuclear fraction) blood cells, with no prior purification of CD₃₄⁺ cells.

PCT/IL03/00064, also to Peled et al., filed Jan. 26, 2003, which is incorporated by reference as if fully set forth herein, teaches the ex-vivo expansion and inhibition of hematopoietic cells using conditions and various molecules that interfere with CD38 expression and/or activity and/or with intracellular copper content, for inducing the ex-vivo expansion of hematopoietic cell populations. The small molecules and methods include linear polyamine chelators and their chelates, nicotinamide, a nicotinamide analog, a nicotinamide or a nicotinamide analog derivative or a nicotinamide or a nicotinamide analog metabolite, a PI 3-kinase inhibitor, conditions for reducing a capacity of the hematopoietic cells in responding to retinoic acid, retinoids and/or Vitamin D and reducing the capacity of the cell in responding to signaling pathways involving PI 3-kinase.

PCT/IL2003/00681, also to Peled et al, filed Aug. 17, 2003, which is incorporated by reference as if fully set forth herein, discloses methods of ex-vivo expanding a population of hematopoietic cells present, even as a minor fraction, in hematopoietic cells, without first enriching the cells, while at the same time, substantially inhibiting differentiation of the hematopoietic cells. Cells thus expanded can be used to efficiently provide ex-vivo cultured populations of hematopoietic cells without prior enrichment of the hematopoietic mononuclear cells for cells suitable for hematopoietic cell transplantation, for genetic manipulations for cellular gene therapy, for adoptive immunotherapy, implantation of stem cells for in vivo transdifferentiation, as well as, ex-vivo tissue engineering in cis-differentiation and transdifferentiation settings.

PCT/IL2004/000215, also to Peled et al., filed Mar. 4, 2004, which is incorporated by reference as if fully set forth herein, further demonstrated the self-renewal of stem/early progenitor cells, resulting in expansion and inhibition of differentiation in cells of hematopoietic origin and non-hematopoietic origin by exposure to low molecular weight inhibitors of PI 3-kinase, disruption of the cells' PI 3-K signaling pathways.

PCT/IL2004/000644, also to Peled et al, filed Jul. 15, 2004, which is incorporated by reference as if fully set forth herein, discloses the expansion of endodermal- and non-endodermally derived cells for transplantation and the repopulation of endodermal organs.

PCT/IL2005/00994, also to Peled et al., filed Sep. 15, 2005, which is incorporated by reference as if fully set forth herein, discloses the expansion and inhibition of differentiation of stem cells by co-culture with mesenchymal cells, the isolation of expanded populations of undifferentiated cells capable of self-renewal, and the use of such cells and cell populations for stem cell transplantation, genetic manipulations, for cellular gene therapy, adoptive immunotherapy, tissue engineering, and the like.

PCT/IL2005/00753, also to Peled et al., filed Jul. 14, 2005, which is incorporated by reference as if fully set forth herein, discloses methods of expanding and substantially inhibiting differentiation in a population of stem cells ex-vivo and/or in-vivo, an expanded, large population of undifferentiated, renewable stem cells, and therapeutic uses thereof for hematopoietic cell transplantations, genetic manipulations and cellular gene therapy, adoptive immunotherapy, treatments for multiple diseases, such as, for example, β-hemoglobinopathia, ex vivo tissue engineering and the like.

Thus, methods are available for expansion and inhibition of differentiation of hematopoietic cells, yielding populations of cells characterized by self-renewal, suitable for hematopoietic and other cell transplantation, for genetic manipulations for cellular gene therapy, adoptive immunotherapy, in vivo and ex-vivo cis-differentiation and trans-differentiation, organ repopulation, etc. However, cell therapy has not succeeded in providing an efficacious solution to the problems inherent in conventional drug and surgical treatments for PVD.

### SUMMARY OF THE INVENTION

While reducing to practice, it was shown that hematopoietic cells cultured and expanded using the methods of the present invention were therapeutic when transplanted into subjects suffering from peripheral vascular disease. Thus, according to some aspects of some embodiments the methods of the present invention can be used for the treatment and prevention of peripheral vascular disease by administering to subject in need thereof. According to another embodiment an article of manufacture comprising a population of cultured hematopoietic cells according to the methods of the present invention, packaging material and a label or package insert indicating that the hematopoietic cell population is for treating a peripheral vascular disease in a subject.

According to an aspect of some embodiments of the present invention there is provided a method of treating a peripheral vascular disease in a subject in need thereof, the method including administering a therapeutic amount of ex-vivo expanded hematopoietic cells to an ischemic tissue of said subject, thereby treating said peripheral vascular disease.

According to some embodiments of the invention the hematopoietic cells are expanded by propagation ex-vivo by (a) culturing hematopoietic cells under conditions allowing for cell proliferation and (b) culturing the hematopoietic cells in the presence of an amount of nicotinamide for a culture period resulting in an expanded population of undifferentiated hematopoietic cells capable of enhancing perfusion in an ischemic tissue of the subject in need thereof.

According to an aspect of some embodiments of the present invention there is provided a method of treating an ischemic disease or condition in a subject in need thereof, the method including administering to a subject in need thereof a therapeutic amount of hematopoietic cells propagated ex-vivo by (a) culturing hematopoietic cells under conditions allowing for cell proliferation and (b) culturing the hematopoietic cells in the presence of an amount of nicotinamide for a culture period resulting in an expanded population of undifferentiated hematopoietic cells capable of enhancing perfusion in an ischemic tissue of the subject in need thereof, and (c) administering a therapeutic amount of the expanded hematopoietic cells to the subject, thereby treating and/or preventing the ischemic disease in the subject.

According to some embodiments of the invention the method further includes enriching the expanded hematopoietic cells for hematopoietic stem cells before the administering to the subject.

According to some embodiments of the invention the administering is effected by a method selected from the group consisting of intravenous administration and direct infusion.

According to some embodiments of the invention the subject is treated with immunosuppressive treatment prior to the administration of the hematopoietic cells and/or following the administration of the hematopoietic cells.

According to some embodiments of the invention the administering includes at least two administrations of the cells to the subject.

According to some embodiments of the invention the expanded hematopoietic cells are co-administered in conjunction with an additional treatment for peripheral vascular disease. The additional treatment can be selected from the group consisting of immunosuppressive treatment, antihypertensive treatment and antiplatelet treatment.

According to some embodiments of the invention the enhanced perfusion is determined according to a parameter selected from the group consisting of Doppler ultrasound, angiogography and MRI.

According to some embodiments of the invention the enhanced perfusion is determined according to a clinical parameter selected from the group consisting of tissue necrosis, tissue ulceration, digit amputation and limb amputation.

According to an aspect of some embodiments of the present invention there is provided a method of preparing hematopoietic cells for administration to a subject suffering from an ischemic disease or condition, the method including (a) culturing hematopoietic cells under conditions allowing for cell proliferation and (b) culturing the hematopoietic cells in the presence of an amount of nicotinamide for a culture period resulting in an expanded population of undifferentiated hematopoietic cells capable of enhancing perfusion in an ischemic tissue of the subject in need thereof. According to some embodiments of the invention the ischemic disease or condition is a peripheral vascular disease.

According to an aspect of some embodiments of the present invention there is provided an article of manufacture for treatment of peripheral vascular disease comprising a packaging material and an ex-vivo cultured hematopoietic cell population, the hematopoietic cell population propagated ex-vivo by (a) culturing hematopoietic cells under conditions allowing for cell proliferation and (b) culturing the hematopoietic cells in the presence of an amount of nicotinamide for a culture period resulting in an expanded population of undifferentiated hematopoietic cells capable of enhancing perfusion in an ischemic tissue and wherein the packaging material comprises a label, instructions or a package insert indicating that the hematopoietic cell population is for treating a peripheral vascular disease in a subject in need thereof.

According to some embodiments of the invention the hematopoietic cells are from a source selected from the group consisting of bone marrow, peripheral blood and neonatal umbilical cord blood.

According to some embodiments of the invention the method further includes enriching the hematopoietic cells for hematopoietic stem cells before the culturing.

According to some embodiments of the invention the method further comprises enriching the expanded hematopoietic cells for hematopoietic stem cells before the culturing.

According to some embodiments of the invention the ischemic disease or condition is a peripheral vascular disease.

According to some embodiments of the invention the amount of nicotinamide is about 0.1 mM to about 20 mM.

According to some embodiments of the invention the amount of nicotinamide is about 0.25 mM to about 15 mM.

According to some embodiments of the invention the amount of nicotinamide is about 0.5 mM to about 10 mM.

According to some embodiments of the invention the amount of nicotinamide is about 1.0 mM to about 10 mM.

According to some embodiments of the invention the amount of nicotinamide is about 5.0 mM.

According to some embodiments of the invention the culture period is about 6 days to about 6 weeks.

According to some embodiments of the invention the culture period is about 10 days to about 5 weeks.

According to some embodiments of the invention the culture period is about 2 weeks to about 4 weeks.

According to some embodiments of the invention the culture period is about 3 weeks.

According to some embodiments of the invention the amount of nicotinamide is 5.0 mM/L and the culture period is about 3 weeks.

According to some embodiments of the invention the conditions for proliferation include providing cytokines.

According to some embodiments of the invention the cytokines are early acting cytokines.

According to some embodiments of the invention the early acting cytokines are selected from the group consisting of stem cell factor, FLT3 ligand, interleukin-1, interleukin-2, interleukin-3, interleukin-6, interleukin-10, interleukin-12, tumor necrosis factor-α and thrombopoietin.

According to some embodiments of the invention the method further includes providing late acting cytokines.

According to some embodiments of the invention the late acting cytokines are selected from the group consisting of: granulocyte colony stimulating factor, granulocyte/macrophage colony stimulating factor, erythropoietin, FGF, EGF, NGF, VEGF, LIF, Hepatocyte growth factor and macrophage colony stimulating factor.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a graphic representation of the hind-limb ischemia model of Peripheral Vascular Disease (PVD) used in the Examples. An arrow indicates the position of ligation of the femoral artery. Note that the cells were administered intramuscularly, distal to the ligated portion of the limb;

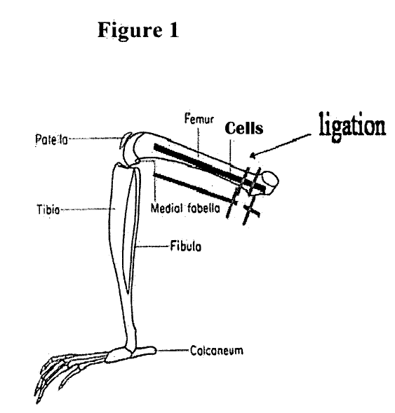

FIG. 2 is a histogram showing the effect of infusion of increasing doses (5×10⁴, 5×10⁵ and 2.5×10⁶ cells per mouse) of cultured and expanded hematopoietic cells on outcome (amputation) in the hind limb ischemia model of immunocompetent BalbC mice. Note the significant prevention (>40%) of ischemia effects with low dose, and near total reversal of ischemia effects in the high dose groups;

FIGS. 3a to 3b are histograms showing the effect of increasing doses (5×10⁴, 5×10⁵ and 2.5×10⁶ cells per mouse) of infused cultured and expanded hematopoietic cells on outcome (perfusion) in the hind limb ischemia model of immunocompetent BalbC mice, measured at day 7 post-ligation (FIG. 3a) and day 14 post ligation (FIG. 3b). Note the rapid, and significantly superior recovery of the treated limbs in all dosage groups, compared to the untreated group. The results are expressed as the percent of change in perfusion, (change in perfusion=perfusion_{day7}-perfusion_{day0}). At each time point percent perfusion is calculated relative to the contralateral, non-ischemic limb;

FIGS. 4a to 4b are a series of laser Doppler blood-flow images showing the effect of infusion of expanded hematopoietic cells on limb perfusion following ligation. Laser Doppler scans of mice was performed 7 days after left hind limb iliac artery ligation in mice injected with cyclosporine (FIG. 4a) or cyclosporine and 5×10⁵ cultured cells. Representative mice of each group are shown. (4b).

A red hue indicates regions with maximum perfusion; a yellow hue indicates intermediate perfusion (values are shown in yellow) and the lowest perfusion values are represented as blue;

FIGS. 5a and 5b are a series of laser Doppler blood-flow images showing the effect of infusion of expanded hematopoietic cells on limb perfusion following ligation. Laser Doppler scans of mice were performed 7 days after left hindlimb iliac artery ligation in mice injected with buffer (FIG. 5a) or 5×10⁵ cells (FIG. 5b). Representative mice are shown. A red hue indicates regions with maximum perfusion; a yellow hue indicates intermediate perfusion (values are shown in yellow) and the lowest perfusion values are represented as blue;

FIG. 6 is a histogram showing the therapeutic effect of infused fresh or cultured and expanded hematopoietic stem cells (1×10⁶ per mouse) on outcome (perfusion) in the hind limb ischemia model in Nude mice, measured at 12 days post-ligation. Note the superior recovery of blood flow of ischemic limbs of mice treated with Nicotinamide cultured cells. The results of this experiment demonstrate the superior activity of Nicotinamide cultured cells over the activity of similar number of cells cultured without nicotinamide or similar number of cells before culture (non-cultured cells). Thus, cells cultured in the presence of Nicotinamide clearly display increased therapeutic potential over cultured or fresh, non-cultured cells;

FIG. 7 is a histogram showing the effect of infused fresh (non-cultured) or cultured and expanded hematopoietic stem cells (1×10⁶ per mouse) on digit and/or limb amputation in the hind limb ischemia model of Nude mice, measured at 12 days post ligation. Note the complete protection from the effects of hind limb ischemia afforded by the nicotinamide-cultured hematopoietic cells.

### DESCRIPTION OF EMBODIMENTS OF THE INVENTION

The present invention, in some embodiments thereof, relates to cell therapy of peripheral vascular disease and, more particularly, but not exclusively, to ex-vivo cultured hematopoietic cells and their use for the treatment of peripheral vascular disease.

The principles and operation of the present invention may be better understood with reference to the accompanying descriptions and examples.

Before explaining at least one embodiment of the invention in detail, it is to be understood that the invention is not limited in its application to the details of construction and the arrangement of the components set forth in the following description or illustrated in the Examples section. The invention is capable of other embodiments or of being practiced or carried out in various ways. Also, it is to be understood that the phraseology and terminology employed herein is for the purpose of description and should not be regarded as limiting.

Peripheral vascular disease (PVD, also known as peripheral arterial occlusive disease, PAOD) is a complex, potentially debilitating and often untreatable condition causing growing concern among greater numbers of young and old individuals. Due to the chronic, and most often irreversible nature of the vascular injury involved, neoangiogenesis has been the goal of many recently proposed solutions for PVD.

While reducing some embodiments of the present invention to practice, it was shown that hematopoietic cells cultured using the methods of the present invention were effective in enhancing perfusion and decreasing extremity amputation when transplanted into subjects suffering from peripheral vascular disease.

Thus, according to one aspect of the present invention there is provided a method of treating a peripheral vascular disease in a subject in need thereof, the method comprising administering a therapeutic amount of ex-vivo expanded hematopoietic cells to an ischemic tissue of the subject, thereby treating the peripheral vascular disease.

As used herein, “peripheral vascular disease (PVD, also known as peripheral occlusive arterial disease, POAD)” comprises conditions resulting from impairment of peripheral circulation (i.e. blood vessels outside those of the heart and brain), for example, as a result of disease or injury, such as inflammation, diabetes, coronary artery disease, myocardial infarction (MI), atrial fibrillation, transient ischemic attack, stroke, limb ischemia and renal disease. Particularly, peripheral vascular disease is a vascular disorder involving blockage in the carotid or femoral arteries, iliac artery, or in arteries distal to the femoral or carotid arteries, such as the tibial artery and it's branches in the lower limb and the brachial artery and it's branches in the upper limb. Also affected are the smaller blood vessels such as the capillary beds of the tissues distal to the blockage. Blockage in the peripheral arteries causes pain and restricted movement, loss of vitality and anoxia of the affected tissue, and may result in necrosis and secondary infection. A specific disorder associated with occlusive peripheral vascular disease is diabetic foot, which affects diabetic patients, often resulting in amputation of the foot.

As used herein, the term “ischemia” refers to partial or complete cessation of blood flow to a tissue.

As used herein, the phrase “subject in need thereof” refers to an individual who has been diagnosed with a peripheral vascular disease, whether overtly symptomatic or seemingly without symptoms, and/or an individual at risk of developing symptoms of peripheral vascular disease.

As used herein, the phrase “hematopoietic stem cells” refers to pluripotent cells that, given the right growth conditions, may develop to any blood cell lineage present in the organism from which they were derived. The phrase, as used herein, refers both to the earliest renewable cell population responsible for generating blood tissue and the very early myeloid and lymphoid progenitor cells, which are somewhat more differentiated, yet are not committed and can readily revert to become a part of the earliest renewable cell population. Hematopoietic stem and progenitor cells are commonly identified according to the presence of markers such as CD34 and CD133, and the absence of other differentiation markers, such as CD38 and various lineage (Lin) markers. Methods of ex-vivo culturing stem cells of different tissue origins are well known in the art of cell culturing. To this effect, see for example, the text book “Culture of Animal Cells—A Manual of Basic Technique” by Freshney, Wiley-Liss, N. Y. (1994), Third Edition, the teachings of which are hereby incorporated by reference.

The phrase “cell expansion” is used herein to describe a process of cell proliferation substantially devoid of cell differentiation. In some embodiments of the present invention, culturing cells with inhibitors of cellular differentiation results in expansion of specific, undifferentiated sub-sectors of the initial cell populations taken for expansion, such as the CD34+, CD133+, CD34+/CD38−, CD34+/Lin− cell types considered advantageous and desirable for cell therapy of PVD. Expansion of such hematopoietic stem and/or progenitor cells can be monitored throughout culturing by detection of appropriate cell markers, as further detailed hereinunder.

As used herein the term “ex-vivo” refers to a process in which cells are removed from a living organism and are propagated outside the organism (e.g., in a test tube, in a culture bag, in a bioreactor). As used herein, the term “ex-vivo”, however, does not refer to a process by which cells known to propagate only in-vitro, such as various cell lines (e.g., HL-60, MEL, HeLa, etc.) are cultured. In other words, cells cultured ex-vivo according to some embodiments of the present invention do not transform into cell lines in that they eventually undergo differentiation. Providing the ex-vivo grown cells with conditions for ex-vivo cell proliferation include providing the cells with nutrients and preferably with one or more cytokines, as is further detailed hereinunder.

As used herein the term “differentiation” refers to relatively generalized or specialized changes during development. Cell differentiation of various lineages is a well-documented process and requires no further description herein. As used herein the term differentiation is distinct from maturation and senescence, which is a process, although sometimes associated with cell division, in which a specific cell type matures to function and then dies, e.g., via programmed cell death.

As used herein, the term “treating” includes abrogating, substantially inhibiting, slowing or reversing the progression of a disease or condition, substantially ameliorating clinical or aesthetical symptoms of a disease or condition or substantially preventing the appearance of clinical, functional or aesthetic symptoms of a disease or condition.

Ex-vivo expansion of hematopoietic cells, under conditions substantially inhibiting differentiation, has been described, and protocols for the expansion of hematopoietic cells by culturing with the polyamine copper chelator TEPA, or by culturing with a PI 3-kinase inhibitor, which are incorporated herein by reference, are described further below. For example, PCT IL03/00064 to Peled et al, which is incorporated by reference as if fully set forth herein, teaches methods of reducing expression and/or activity of CD38 in cells, methods of reducing capacity of cells in responding to signaling pathways involving CD38 in the cells, methods of reducing capacity of cells in responding to retinoic acid, retinoids and/or Vitamin D in the cells, methods of reducing the capacity of cells in responding to signaling pathways involving the retinoic acid receptor, the retinoid X receptor and/or the Vitamin D receptor in the cells, methods of reducing the capacity of cells in responding to signaling pathways involving PI 3-kinase, conditions wherein cells are cultured in the presence of nicotinamide, a nicotinamide analog, a nicotinamide or a nicotinamide analog derivative or a nicotinamide or a nicotinamide analog metabolite and conditions wherein cells are cultured in the presence of a PI 3-kinase inhibitor.

According to presently known embodiments of this aspect of the present invention, nicotinamide is used as an effective CD38 inhibitor. Hence, in one embodiment, the method according to this aspect of the present invention is effected by providing the cells either with nicotinamide itself, or with a nicotinamide analog, a nicotinamide or a nicotinamide analog derivative or a nicotinamide or a nicotinamide analog metabolite.

As used herein, the phrase “nicotinamide analog” refers to any molecule that is known to act similarly to nicotinamide. Representative examples of nicotinamide analogs include, without limitation, benzamide, nicotinethioamide (the thiol analog of nicotinamide), nicotinic acid and α-amino-3-indolepropionic acid.

The phrase “a nicotinamide or a nicotinamide analog derivative” refers to any structural derivative of nicotinamide itself or of an analog of nicotinamide. Examples of such derivatives include, without limitation, substituted benzamides, substituted nicotinamides and nicotinethioamides and N-substituted nicotinamides and nicotinthioamides.

The phrase “a nicotinamide or a nicotinamide analog metabolite” refers to products that are derived from nicotinamide or from analogs thereof such as, for example, NAD, NADH and NADPH.

Hematopoietic cells can be expanded in culture using a variety of conditions. The ex-vivo expansion and preparation of the hematopoietic cells for transplantation for treatment of peripheral vascular disease is performed according to a protocol including parameters resulting in expanded hematopoietic cells having greater capability of enhancing perfusion in ischemic tissues associated with peripheral vascular occlusion and/or peripheral vascular disease. In addition, hematopoietic cells ex-vivo expanded as described herein can used for treatment of additional ischemic disease or conditions not restricted to peripheral vascular disease.

In some embodiments, the hematopoietic cells are cultured in the presence of an amount of nicotinamide or the analogs, derivatives or metabolites thereof within final concentrations in the millimolar ranges. For example, nicotinamide concentration can be about 0.075 mM, about 0.1 mM, about 0.125 mM, about 0.15 mM, about 0.2 mM, about 0.25 mM, about 0.3 mM, about 0.35 mM, about 0.4 mM, about 0.45 mM, about 0.5 mM, about 0.6 mM, about 0.7 mM, about 0.8 mM, about 0.9 mM, about 1.0 mM, about 1.25 mM, about 1.5 mM, about 2.0 mM, about 2.5 mM, about 3.0 mM, about 3.5 mM, about 4.0 mM, about 4.5 mM, about 5.0 mM, about 6.0 mM, about 7.0 mM, about 8.0 mM, about 9.0 mM, about 10.0 mM, about 11 mM, about 12 mM, about 13 mM, about 14 mM, about 15 mM, about 16 mM, about 17 mM, about 18 mM, about 19 mM, about 20 mM or more, or within about 0.1 mM to about 20 mM, about 0.25 mM to about 15 mM, about 0.5 mM to about 10 mM, within 1.0 mM to about 10 mM, within 1.0 mM to about 5 mM. As used herein the term “about” refers to ±10% of the indicated value. In an exemplary embodiment the nicotinamide concentration is about 5 mM/L.

In some embodiments, nicotinamide or the analogs, derivatives or metabolites thereof are provided for a culture period of between a few days to a number of weeks, for example, 6 days, 7 days, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 4 weeks, 29 days, 30 days, 31 days, 32 days, 33 days, 34 days, 5 weeks, 38 days, 40 days, 6 weeks, 45 days, 7 weeks, or more, or within about 6 days to about 5 weeks, about 10 days to about 4 weeks, about 2 weeks to about 3 weeks. In one exemplary embodiment, the culture period is about 3 weeks.

Further, according to some embodiments of the present invention, ex-vivo expansion of the hematopoietic cells comprises providing the cells with the conditions for ex-vivo cell proliferation such as nutrients and with cytokines. The cytokines can be early acting cytokines, such as, but not limited to, stem cell factor, FLT3 ligand, interleukin-1, interleukin-2, interleukin-3, interleukin-6, interleukin-10, interleukin-12, tumor necrosis factor-α and thrombopoietin. In an exemplary embodiment, the hematopoietic cells are cultured in the presence of a combination of stem cell factor (SCF), thrombopoietin (TPO), IL-6 and FLT3 ligand.

Late acting cytokines can also be used. These include, for example, granulocyte colony stimulating factor, granulocyte/macrophage colony stimulating factor, erythropoietin, FGF, EGF, NGF, VEGF, LIF, Hepatocyte growth factor and macrophage colony stimulating factor.

It will be appreciated in this respect that novel cytokines are continuously discovered, some of which may find uses in the methods of cell expansion of the present invention.

The hematopoietic cells used in the present invention can be of various origin. According to an exemplary embodiment of the present invention, the hematopoietic cells are derived from a source selected from the group consisting of hematopoietic cells, umbilical cord blood cells, mobilized peripheral blood cells, bone marrow cells and also from predominately non-hematopoietic tissue such as hepatic cells, pancreatic cells, neural cells, oligodendrocyte cells, skin cells, embryonal stem cells, muscle cells, bone cells, mesenchymal cells, chondrocytes and stroma cells. Methods of preparation of hematopoietic stem cells from a variety of sources are well known in the art, commonly selecting cells expressing one or more hematopoietic cell markers such as CD34, CD133, etc, or lacking markers of differentiated cells. Selection is usually by FACS, or immunomagnetic separation, but can also be by nucleic acid methods such as PCR (see Materials and Experimental Methods hereinbelow). Embryonic hematopoietic cells and methods of their retrieval are well known in the art and are described, for example, in Trounson A O (Reprod Fertil Dev (2001) 13: 523), Roach M L (Methods Mol Biol (2002) 185: 1), and Smith A G (Annu Rev Cell Dev Biol (2001) 17:435). Adult hematopoietic cells can be derived from tissues of adults and are also well known in the art. Methods of isolating or enriching for adult cells are described in, for example, Miraglia, S. et al. (1997) Blood 90: 5013, Uchida, N. et al. (2000) Proc. Natl. Acad. Sci. USA 97: 14720, Simmons, P. J. et al. (1991) Blood 78: 55, Prockop D J (Cytotherapy (2001) 3: 393), Bohmer R M (Fetal Diagn Ther (2002) 17: 83) and Rowley S D et al. (Bone Marrow Transplant (1998) 21: 1253), Stem Cell Biology Daniel R. Marshak (Editor) Richard L. Gardner (Editor), Publisher: Cold Spring Harbor Laboratory Press, (2001) and Hematopoietic Stem Cell Transplantation. Anthony D. Ho (Editor) Richard Champlin (Editor), Publisher: Marcel Dekker (2000).

PCT IL03/00681 to Peled, et al, which is incorporated by reference as if fully set forth herein, discloses the expansion use of molecules which are capable of repressing differentiation and stimulating and prolonging proliferation of hematopoietic stem cells when the source of cells includes the entire fraction of mononuclear blood cells, namely non-enriched cells. Thus, in one embodiment of the present invention, the population of cells comprising hematopoietic cells is unselected mononuclear cells.

The phrase “unselected hematopoietic mononuclear cells” is used herein to describe any portion of the white blood cells fraction, in which the majority of the cells are hematopoietic committed cells, while the minority of the cells are hematopoietic stem and progenitor cells, as these terms are further defined hereinunder. In a healthy human being, the white blood cells comprise a mixture of hematopoietic lineage committed and differentiated cells (typically over 99% of the mononuclear cells are lineage committed cells) including, for example: Lineage committed progenitor cells CD34⁺CD33⁺ (myeloid committed cells), CD34⁺CD3⁺ (lymphoid committed cells) CD34⁺CD41⁺ (megakaryocytic committed cells) and differentiated cells—CD34⁻CD33⁺ (myeloids, such as granulocytes and monocytes), CD34⁻CD3⁺, CD34⁻CD19⁺ (T and B cells, respectively), CD34⁻CD41⁺ (megakaryocytes), and hematopoietic stem and early progenitor cells such as CD34⁺Lineage negative (Lin⁻), CD34-Lineage negative CD34⁺CD38⁻ (typically less than 1%).

Hematopoietic mononuclear cells are typically obtained from a blood sample by applying the blood sample onto a Ficoll-Hypaque layer and collecting, following density-cushion centrifugation, the interface layer present between the Ficoll-Hypaque and the blood serum, which interface layer essentially consists of the white blood cells present in the blood sample.

Hematopoietic stem and progenitor cells can be obtained by further selection or enrichment of the hematopoietic mononuclear cells obtained by differential density centrifugation as described above. This further enrichment process is typically performed by immuno-separation such as immunomagnetic- separation or FACS and results in a cell fraction that is enriched for hematopoietic cells (for detailed description of enrichment of hematopoietic cells, see Materials and Experimental Procedures in the Examples section).

Enrichment by selection according to the abovementioned markers can be performed prior to expansion of the hematopoietic cells, during the period of culturing, or following expansion and prior to administration of the cells to the recipient. In another embodiment, unselected hematopoietic mononuclear cells can be used as a direct source for obtaining expanded population of hematopoietic cells, circumventing the need for stem cell enrichment prior to expansion, thereby substantially simplifying the process in terms of both efficiency and cost.

It will be appreciated that establishment of protocol for treatment may entail calibration of parameters such as nicotinamide concentration, duration of culture period, amount of cells administered per dose, number of doses, and the like. The influence of such parameters can be evaluated by routine experimentation, such as the models described in the Examples section below. Outcome measures such as perfusion and survival, as well as histological and functional criteria, can be employed to assess the efficacy of varying the different parameters, in order to approach optimal efficiency in numbers of cells having maximal therapeutic value in treating peripheral vascular disease. Additional parameters known in the art that can be quantified for determining perfusion in an affected tissue are angiography and MRI, and clinical parameters such as extent of tissue necrosis in the affected area, tissue ulceration in the ischemic area, and amputation of digits and/or limbs.

The data obtained from these animal studies can be used in formulating a range of dosage for use in human. For example, therapeutically effective doses suitable for treatment of peripheral vascular disease can be determined from the experiments with animal models of these diseases.

The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide numbers of hematopoietic cells which are sufficient to maintain the modulating effects. In one embodiment the subject is treated with a single dose of hematopoietic cells. In another embodiment, administering the hematopoietic cells includes at least two administrations or more of cells to the subject.

According to some embodiments of the present invention, expanded hematopoietic cells are administered by implantation into a subject. Methods of cellular therapy, that is, transplanting hematopoietic cells into a recipient are well know in the art (see, for example, the numerous references in the Background section hereinabove). Suitable methods of transplantation can be determined by monitoring the homing of the implanted cells to a desired tissue, the expression of desired tissue-specific genes or markers, and the function of the transplanted tissue in the recipient. In some embodiments, the expanded hematopoietic are administered locally into the area, organ, tissue or limb affected by the ischemic processes of the peripheral vascular disease by direct infusion into the tissue. Such local administration can be, for example, but not exclusively, using a hypodermic needle or catheter for intra-muscular administration, or via intravascular administration to the effected region.

Hematopoietic cells for administration according to the methods of the present invention are prepared in a suitable medium for transplantation. Such a medium may include a pharmaceutically acceptable vehicle such as a physiological buffer solution such as PBS or Ringers solution, or the medium may be supplemented with additional factors such as nutrients, vitamins, electrolytes, etc, as determined by the administering physician or individual.

The donor and the recipient of the expanded hematopoietic cells can be a single individual or different individuals, for example, allogeneic individuals. When allogeneic transplantation is practiced, regimes for reducing implant rejection and/or graft vs. host disease, as well know in the art, should be undertaken. Such regimes are currently practiced in human therapy. Most advanced regimen are disclosed in publications by Slavin S. et al., e.g., J Clin Immunol (2002) 22: 64, and J Hematother Stem Cell Res (2002) 11: 265), Gur H. et al. (Blood (2002) 99: 4174), and Martelli M F et al, (Semin Hematol (2002) 39: 48), which are incorporated herein by reference.

In order to provide additional therapeutic value to the treated ischemic tissues, the expanded hematopoietic cells of the present invention can be genetically modified to express a factor or factors effective in combating ischemic damage in PVD, i.e. gene therapy. Gene therapy as used herein refers to the transfer of genetic material (e.g., DNA or RNA) of interest into a host to treat or prevent a genetic or acquired disease or condition or phenotype. The genetic material of interest encodes a product (e.g., a protein, polypeptide, peptide, functional RNA, antisense) whose production in vivo is desired. For example, the genetic material of interest can encode a hormone, receptor, enzyme, polypeptide or peptide of therapeutic value. For review see, in general, the text “Gene Therapy” (Advanced in Pharmacology 40, Academic Press, 1997).

The cells can be genetically modified ex-vivo, or can be derived from a genetically modified tissue or host. In ex-vivo gene therapy cells are removed from a patient, and while being cultured are treated in-vitro. Generally, a functional replacement gene is introduced into the cells via an appropriate gene delivery vehicle/method (transfection, transduction, homologous recombination, etc.) and an expression system as needed and then the modified cells are expanded in culture and returned to the host/patient. These genetically re-implanted cells have been shown to express the transfected genetic material in situ.

Hence, further according to an aspect of the present invention, the expanded hematopoietic cells are genetically modified cells. Methods for transducing expanded hematopoietic cells with a transgene are known in the art (see, for example, PCT IL2004/000215 to Peled, incorporated herein by reference). Briefly, a nucleic acid molecule introduced into the hematopoetic cell in a form suitable for expression in the cell of the gene product encoded by the nucleic acid. Accordingly, the nucleic acid molecule includes coding and regulatory sequences required for transcription of a gene (or portion thereof) and, when the gene product is a protein or peptide, translation of the gene acid molecule include promoters, enhancers and polyadenylation signals, as well as sequences necessary for transport of an encoded protein or peptide, for example N-terminal signal sequences for transport of proteins or peptides to the surface of the cell or secretion.

Gene products suitable for use with the methods of the present invention can include, but not exclusively, pro-angiogenic factors such as VEGF, PDGF, FGF, D114, MMP, Ang1, Ang2, anti-inflammatory factors such as the anti-inflammatory cytokines IL-4, IL-6, IL-10, IL-11, IL-13 and the like. Providing such factors through transplantation of expanded hematopoietic cells can serve to augment neoangiogenesis in the damaged tissue and hasten recovery from PVD-related ischemia.

The method of the present invention can be administered along with other therapies for peripheral vascular disease, such as an adjunct therapy or in co-administration. Thus, in one embodiment, the expanded hematopoietic cells are co-administered in conjunction with an additional treatment affecting peripheral vascular disease, such as anti-inflammatory treatment, antiseptic and antibiotic treatment, hormonal replacement such as insulin, estrogen and the like, palliative treatment such as anti-nociceptive treatment, statins and anticoagulative and thrombolytic treatment such as heparin and coumadin. Co-adminstration may be performed by actually administering the additional therapy simultaneously with the expanded hemtopoietic cells, or, more likely, by administering the additional therapy for a period of time close to, during or soon after the administration of the expanded hematopoietic cells.

As used herein the terms “regeneration of blood vessels,” angiogenesis,” “neo-angiogenesis” “revascularization,” and “increased collateral circulation” (or words to that effect) are considered as synonymous. The term “pharmaceutically acceptable” when referring to a natural or synthetic substance means that the substance has an acceptable toxic effect in view of its much greater beneficial effect, while the related the term, “physiologically acceptable,” means the substance has relatively low toxicity. The term, “co-administered” means two or more drugs are given to a patient at approximately the same time or in close sequence so that their effects run approximately concurrently or substantially overlap. This term includes sequential as well as simultaneous drug administration.

As mentioned hereinabove, there are other methods available for expansion of hematopoietic cells in culture, such as inhibitors of activity or expression of PI 3-kinase. PCT IL2004/000215 to Peled et al., which is incorporated by reference as if fully set forth herein, discloses the use of inhibitors of PI 3-K activity or expression for ex-vivo expansion of stem and/or progenitor cells while inhibiting differentiation thereof.

Thus, in still another particular embodiment of this aspect of the present invention, culturing the stem and/or progenitor cells ex-vivo under conditions allowing for cell proliferation and at the same time inhibiting differentiation is effected by culturing the cells in conditions reducing the capacity of the cells in responding to signaling pathways involving PI 3-kinase, or in conditions wherein the cells are cultured in the presence of the PI 3-kinase inhibitors.

In some embodiments, inhibition of PI 3-kinase activity can be effected by known PI 3-kinase inhibitors, such as wortmannin and LY294002 and the inhibitors described in, for example, U.S. Pat. No. 5,378,725, which is incorporated herein by reference.

Final concentrations of the antagonists may be, depending on the specific application, in the micromolar or millimolar ranges. For example, within about 0.1 μM to about 100 mM, preferably within about 4 μM to about 50 mM, more preferably within about 5 μM to about 40 mM.

In still another particular embodiment of this aspect of the present invention, culturing the hematopoietic cells ex-vivo under conditions allowing for cell proliferation and at the same time inhibiting differentiation is effected by culturing the cells in the presence of a copper chelator. Detailed description of transition metal chelators having high affinity for copper suitable for efficient ex-vivo expansion of hematopoietic cells, while substantially inhibiting differentiation thereof in the present invention can be found in PCT IL99/00444, PCT IL 03/00064, PCT IL 03/00681, PCT IL 2004/000215, and PCT IL2005/00994 to Peled, et al, which are incorporated by reference as if fully set for herein.

In yet another particular embodiment of this aspect of the present invention, culturing the hematopoietic cells ex-vivo under conditions allowing for cell proliferation and at the same time inhibiting differentiation is effected by culturing the cells in the presence of a copper chelate (chelator-Cu complex). PCT IL03/00062 to Peled, et al, which is incorporated by reference as if fully set for herein, discloses the use of copper chelates, complexes of copper and heavy metal chelators having high affinity for copper, for efficient ex-vivo expansion of hematopoietic cells, while substantially inhibiting differentiation thereof.

Final concentrations of the chelator may be, depending on the specific application, in the micromolar or millimolar ranges. For example, within about 0.1 μM to about 100 mM, preferably within about 4 μM to about 50 mM, more preferably within about 5 μM to about 40 mM.

In some embodiments, copper chelators include polyamine molecules, which can form a cyclic complex with the copper ion via two or more amine groups present in the polyamine. The polyamine chelator can be a linear polyamine, a cyclic polyamine or a combination thereof.

Expanded hematopoietic cell populations of the present invention may, if desired, be presented in a pack or dispenser device, such as FDA approved kit, which may contain one or more unit dosage forms containing the active ingredient. The pack may, for example, comprise metal or plastic foil, such as a blister pack. The pack or dispenser device may be accompanied by instructions for administration for treatment of peripheral vascular disease. The pack or dispenser may also be accompanied by a notice associated with the container in a form prescribed by a governmental agency regulating the manufacture, use or sale of pharmaceuticals, which notice is reflective of approval by the agency of the form of the compositions or human or veterinary administration. Such notice, for example, may be of instructions or labeling approved by the U.S. Food and Drug Administration for prescription drugs or of an approved product insert. Compositions comprising the expanded hematopoietic cells of the invention formulated in a compatible pharmaceutical carrier may also be prepared, placed in an appropriate container, and labeled for treatment or prevention of an indicated condition or induction of a desired event associated with peripheral vascular disease. Suitable indica on the label may include treatment and/or prevention of a peripheral vascular disease.

As used herein, the singular form “a”, “an” and “the” include plural references unless the context clearly dictates otherwise. For example, the term “a compound” or “at least one compound” may include a plurality of compounds, including mixtures thereof.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate some embodiments of the invention in a non limiting fashion.

Materials and Experimental Methods

### Cells and Cell Processing for Expansion and Infusion:

Cell source: Hematopoietic cells were either hematopoietic stem cells (HSC) or progenitor cells (HPC) from either bone marrow (BM), G-CSF mobilized peripheral blood (MPB) or umbilical cord blood (UCB).

Cell cultures of human hematopoietic cells: Human umbilical cord blood cells were obtained from umbilical cord blood after normal full-term delivery (informed consent was given). MPB, or BM were obtained from donations (informed consent was given). Samples were either used fresh or collected and frozen according to well known cord blood cryopreservation protocol (Rubinstein et al. 1995) within 24 hours postpartum for UCB or according to common practice regarding MPB and BM. Prior to cryopreservation, blood was sedimented for 30 minutes on HESPAN Starch (hydroxyethyl starch) to remove most RBC. Prior to their use, the cells were thawed in Dextran buffer (Sigma, St. Louis, Mo., USA) containing 2.5% human serum albumin (HSA)(Bayer Corp. Elkhart, Ind., USA) and processed as described herein below. Following thawing, where indicated, the leukocyte-rich fraction was harvested and layered on Ficoll-Hypaque gradient (1.077 g/mL; Sigma Inc, St Louis Mo., USA), and centrifuged at 400×g for 30 minutes. The mononuclear cell fraction in the interface layer was then collected, washed three times, and re-suspended in phosphate-buffered saline (PBS) (Biological Industries, Bet HaEmek, Israel) containing 0.5% human serum albumin (HSA) (Bayer Corp. Elkhart, Ind., USA). CD34⁺ cells were isolated and purified by immunomagnetic separation using the “MiniMACS CD34⁺ progenitor cell isolation kit” (Miltenyi Biotec, Auburn, Calif.) according to the manufacturer's recommendations. The purity of the CD34⁺ cells obtained ranged between 95% and 98%, based on Flow Cytometry evaluation. The CD133⁺ cell fraction was purified as follows: Either the mononuclear cell fraction was subjected to two cycles of immuno-magnetic separation using the “MiniMACS CD133 stem cell isolation kit” (Miltenyi Biotec, Auburn, Calif.) or the unfractionated preparation was isolated on the CliniMACS device using CD133⁺ CliniMACS (Miltenyi Biotec, Auburn, Calif.) reagent, accordingly, following the manufacturer's recommendations (in the latter, the Ficoll-Hypaque gradient stage was omitted). The purity of the CD133⁺ populations thus obtained was 80-95%, as evaluated by flow cytometry.

Ex vivo Expansion of Hematopoietic Cells

Peripheral blood, bone marrow or cord blood derived hematopoietic cells or purified hematopoietic stem/progenitor cells (CD34+ or CD133+) were cultured with cytokines and either a PI3K enzyme activity inhibitor (LY294002) or nicotinamide, at the indicated concentrations for the indicated duration of culturing period. Following culturing with these inhibitors of differentiation, the resultant cell population was demonstrated to be enriched with subsets of stem/early progenitor cells, such as CD34+CD38− and CD34+Lin− cells, as compared to cells cultured in the presence of cytokines alone.

### Mouse Hind Limb Ischemia Model

12 week old Balb/C or “nude” mice weighing between 26 g and 30 g were used for this model of peripheral vascular disease. Under short-term anaesthesia, the left femoral artery was exposed, dissected free, and excised (Madeddu, Emanueli et al. 2004) (Couffinhal, Silver et al. 1998; Babiak, Schumm et al. 2004).

### Cell Infusion

The impact of cultured cells administration on therapeutic neovascularization was investigated in a murine model of hindlimb ischemia (Kalka, Masuda et al. 2000; Madeddu, Emanueli et al. 2004). One day after operative excision of one femoral artery, mice receive an intramuscular injection of 1-2000×10³ cells expanded as described. Control groups were injected with the same number of unexpanded cells, or with PBS (Madeddu, Emanueli et al. 2004).

In vivo Perfusion Measurement

Perfusion analysis was be performed after femoral artery ligation, before and 7-14 days after cell transplantation (Babiak, Schumm et al. 2004). Laser Doppler perfusion imaging (Moor Instrument, Wilmington, Del.) was used to record serial blood flow measurements over the course of 14 days postoperatively. In these digital color-coded images, red hues indicate regions with maximum perfusion; medium perfusion values are shown in yellow; lowest perfusion values are represented as blue. In order to exclude inter-individual differences as well as investigator-dependent bias, the ratios between ligated and non-ligated limb were measured in a standardized fashion.

### Experimental Results

### Example I

### Hematopoietic Cells Expanded with PI 3 Kinase Inhibitor Prevent Amputation in Ischemic Limbs of Balb/C Mice

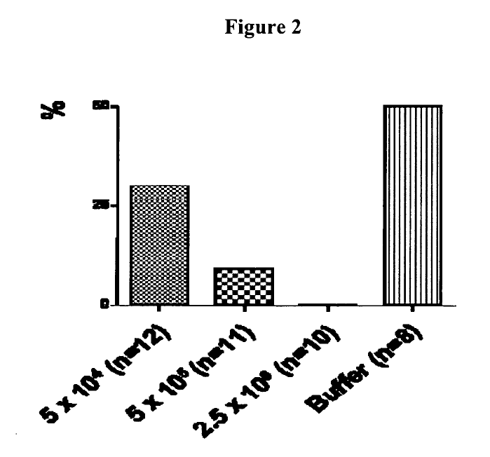

Cord blood derived CD133+ were cultured with cytokines (SCF, TPO, IL-6 and FLT3) and supplemented with the PI3K specific inhibitor LY294002 at a final concentration of 5 micromoles/L. After three weeks in culture the resulting cell population was enumerated, characterized and infused intramuscular in the ischemic limbs of immunocompetent Balb/C mice at different doses as indicated (5×10⁴, 5×10⁵ and 2.5×10⁴ cells per subject). Control mice were infused with buffer. The mice were treated with cyclosporin starting one day before induction of limb ischemia and on. The rate of limb amputation was evaluated 7 days after cell infusion (no amputations occurred after day 7). FIG. 2 shows the dose-dependent prevention of limb amputation in the mice receiving the hematopoietic cells.

### Example II

### Hematopoietic Cells Expanded with PI 3 Kinase Inhibitor Enhance Perfusion in Ischemic Limbs of Balb/C Mice

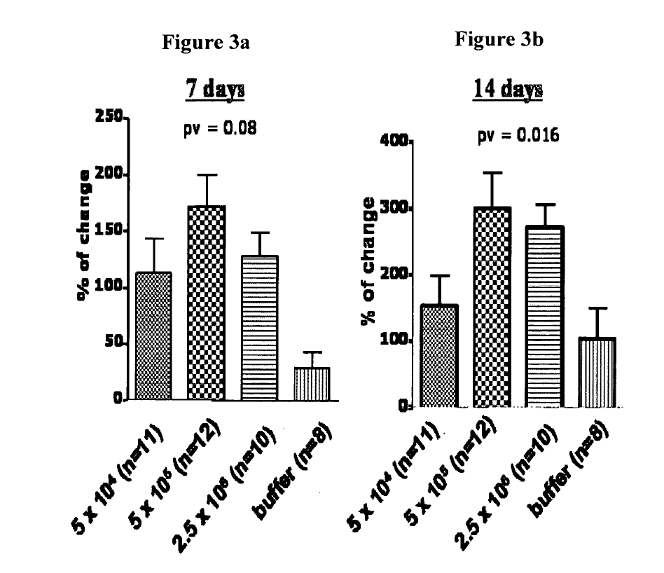

Cord blood derived CD133+ were cultured with cytokines (SCF, TPO, IL-6 and FLT3) and supplemented with the PI-3K inhibitor LY294002 at a concentration of 5 micromoles/L. After three week in culture the resulting cell population was counted, characterized and infused intramuscular into the ischemic limbs of immunocompetent Balb/C mice at the different doses as indicated (5×10⁴, 5×10⁵ and 2.5×10⁴ cells per subject). Control mice were infused with buffer. The mice were treated with cyclosporine starting one day before induction of limb ischemia and afterwards. Limb perfusion was evaluated at day-0 (after induction of ischemia), at day 7 and at day 14. The results are shown as percent perfusion relative to the non-ischemic limb. FIG. 3a shows the dramatic, dose-dependent improvement in limb perfusion 7 days after infusion of the hematopoietic cells. At 14 days after infusion of the hematopoietic cells (FIG. 3b), greatly improved perfusion was observed. Note the rapid and significantly superior recovery of the treated limbs in all dosage groups.

Taken together, these results show that hematopoietic cells, expanded with an inhibitor of PI 3-kinase, even at low doses of 5×10⁴ cells per subject, are able to significantly improve the outcome, rapidly increasing perfusion and reducing the amputation rate, in peripheral vascular arterial occlusion in limbs of immunocompetent hosts.

### Example III

### Hematopoietic Cells Expanded with Nicotinamide Enhance Perfusion in Ischemic Limbs of Balb/C Mice

Cord blood derived CD133+ were cultured with cytokines (SCF, TPO, IL-6 and FLT3) and supplemented with final concentration of 5 mM/L nicotinamide. After three week in culture the resulting cell population was counted, characterized and 5×10⁵ cells per subject were infused intramuscularly into the ischemic limb at different doses as indicated. Control mice were infused with buffer. Limb perfusion was evaluated at day 7 by Doppler Ultrasound. A red hue indicates regions with maximum perfusion; a yellow hue indicates intermediate perfusion (values are shown in yellow) and the lowest perfusion values are represented as blue.

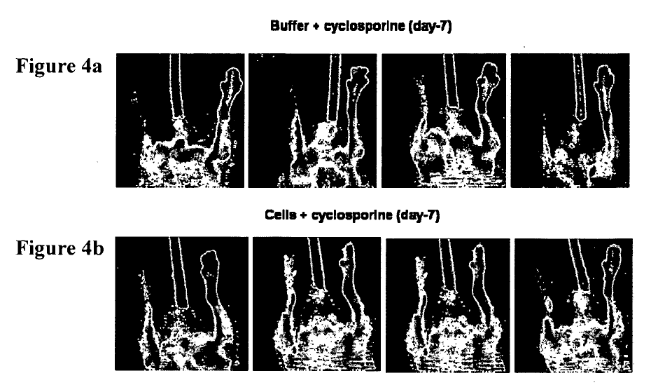

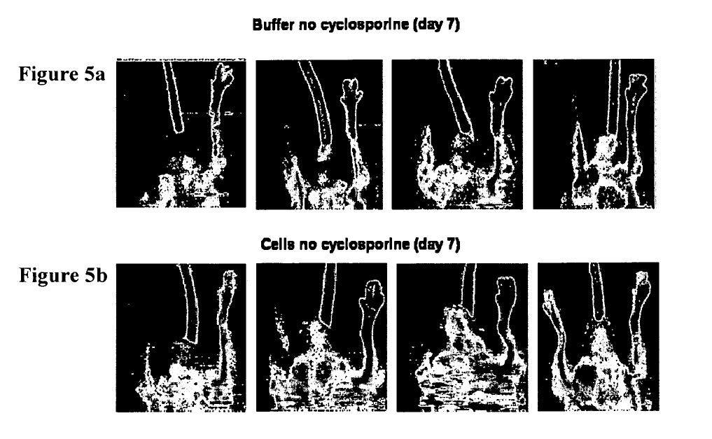

FIGS. 4a and 4b show the improvement in perfusion in the ischemic limb in mice treated with cyclosporine at the day of cell infusion and two additional days therafter. FIGS. 5a and 5b show the improvement in perfusion in the ischemic limb in mice not receiving cyclosporine.

### Example IV

### Hematopoietic Cells Expanded with Nicotinamide Enhance Perfusion in Ischemic Limbs of Nude Mice

Cord blood derived CD133+ were cultured with cytokines (SCF, TPO, IL-6 and FLT3) and supplemented with final concentration of 5 mM/L nicotinamide. After three week in culture the resulting cell population was counted, characterized and 1×10⁶ cells per subject were infused intramuscularly into the ischemic limb of nude (athymic) mice at different doses as indicated. No cyclosporin was administered. Control mice were infused with non-cultured cells or buffer, as indicated. Limb perfusion was evaluated at day 7 by Doppler Ultrasound, and expressed as percent perfusion relative to the contralateral, un-ligated healthy limb in each case.

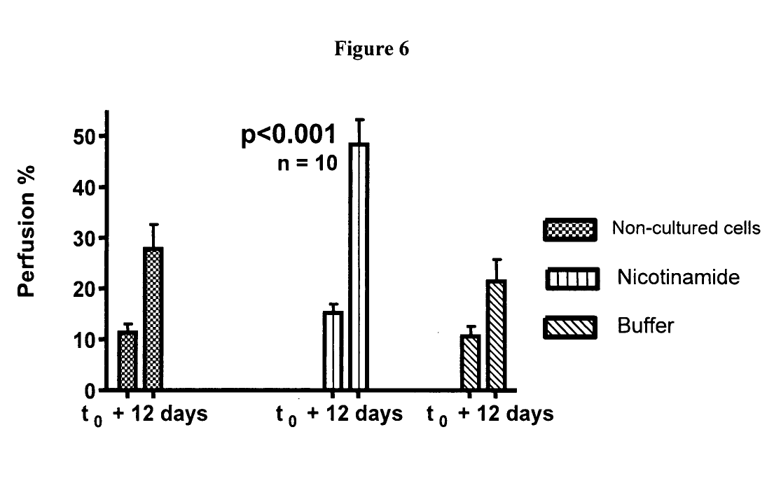

As illustrated in FIG. 6, at 12 days post ligation, while the limbs receiving non-cultured cells showed impaired perfusion similar to that of the untreated (buffer) ligated limbs, ischemic limbs receiving the nicotinamide-expanded cells showed significantly greater recovery of blood flow. The results of this experiment demonstrate the superior activity of Nicotinamide cultured cells over the activity of similar number of cells cultured without nicotinamide or similar number of cells before culture (non-cultured cells). Thus, cells cultured in the presence of Nicotinamide clearly display increased potential for enhancing perfusion in ischemic tissue associated with peripheral vascular disease over cultured or fresh, non-cultured cells;

### Example V

### Hematopoietic Cells Expanded with Nicotinamide Enhance Digit and Limb Survival in Ischemic Limbs of Balb/C Mice

Cord blood derived CD133+ were cultured with cytokines (SCF, TPO, IL-6 and FLT3) and supplemented with final concentration of 5 mM/L nicotinamide. After three week in culture the resulting cell population was counted, characterized and 1×10⁶ cells per subject were infused intramuscularly into the ischemic limb of nude (athymic) mice at different doses as indicated. Control mice were infused with the same amount of non-cultured cells or buffer, as indicated. The rate of limb amputation was evaluated 12 days after cell infusion (no amputation occured after day 12).

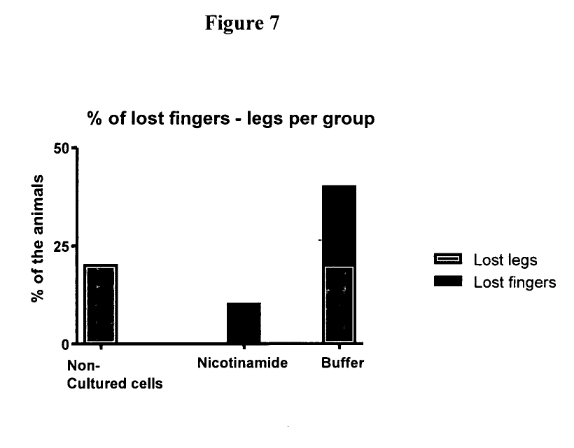

As evidenced by the results shown in FIG. 7, intramuscular infusion of the nicotinamide-expanded cells afforded complete prevention of amputation of the ischemic limb, while limbs receiving fresh, non-cultured cells showed rates of amputation similar to untreated, ischemic limbs.

Taken together, the results presented above indicate that hematopoietic cells expanded in culture with nicotinamide have superior ability to enhance perfusion in ischemic tissue associated with peripheral vascular occlusion, and significantly improve clinical outcome (e.g. limb and digit survival) in the affected limbs. It will be noted that the effects of transfusion of nicotinamide-expanded hematopoietic cells is significant in both immune-competent hosts (Balb/C mice) with and without cyclosporine treatment, and in immune-compromised (athymic) nude mice.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method of treating a peripheral vascular disease in a subject in need thereof, the method comprising administering a therapeutic amount of ex-vivo expanded hematopoietic cells to an ischemic tissue of said subject, thereby treating said peripheral vascular disease.

2. The method of claim 1, wherein said hematopoietic cells are expanded by propagation ex-vivo by:
(a) culturing hematopoietic cells under conditions allowing for cell proliferation and,
(b) culturing said hematopoietic cells in the presence of an amount of nicotinamide for a culture period resulting in an expanded population of undifferentiated hematopoietic cells capable of enhancing perfusion in an ischemic tissue of said subject in need thereof.

3. The method of claim 1, wherein said hematopoietic cells are from a source selected from the group consisting of: bone marrow, peripheral blood and neonatal umbilical cord blood.

4. The method of claim 2, wherein said method further comprises enriching said hematopoietic cells for hematopoietic stem cells before said culturing.

5. The method of claim 1, which further comprises enriching said expanded hematopoietic cells for hematopoietic stem cells before said administering to said subject.

6. The method of claim 2, wherein said amount of nicotinamide is about 0.1 mM to about 20 mM.

7. The method of claim 2, wherein said amount of nicotinamide is about 0.25 mM to about 15 mM.

8. The method of claim 2, wherein said amount of nicotinamide is about 0.5 mM to about 10 mM.

9. The method of claim 2, wherein said amount of nicotinamide is about 1.0 mM to about 10 mM.

10. The method of claim 2, wherein said amount of nicotinamide is about 5.0 mM.

11. The method of claim 2, wherein said culture period is about 6 days to about 6 weeks.

12. The method of claim 2, wherein said culture period is about 10 days to about 5 weeks.

13. The method of claim 2, wherein said culture period is about 2 weeks to about 4 weeks.

14. The method of claim 2, wherein said culture period is about 3 weeks.

15. The method of claim 1, wherein said administering is effected by a method selected from the group consisting of intravenous administration and direct infusion.

16. The method of claim 1, wherein said subject is treated with immunosuppressive treatment prior to said administration of said hematopoietic cells.

17. The method of claim 1, wherein said subject is treated with immunosuppressive treatment following said administration of said hematopoietic cells.

18. The method of claim 1, wherein said administering comprises at least two administrations of said cells to said subject.

19. The method of claim 1, wherein said expanded hematopoietic cells are co-administered in conjunction with an additional treatment for peripheral vascular disease.

20. The method of claim 19, wherein said additional treatment is selected from the group consisting of immunosuppressive treatment, antihypertensive treatment and antiplatelet treatment.

21. The method of claim 2, wherein said enhanced perfusion is determined according to a parameter selected from the group consisting of Doppler ultrasound, angiogography and MRI.

22. The method of claim 2, wherein said enhanced perfusion is determined according to a clinical parameter selected from the group consisting of tissue necrosis, tissue ulceration, digit amputation and limb amputation.

23. The method of claim 2, wherein said conditions for proliferation comprise providing cytokines.

24. The method of claim 23, wherein said cytokines are early acting cytokines.

25. The method of claim 24, wherein said early acting cytokines are selected from the group consisting of: stem cell factor, FLT3 ligand, interleukin-1, interleukin-2, interleukin-3, interleukin-6, interleukin-10, interleukin-12, tumor necrosis factor-α and thrombopoietin.

26. The method of claim 24, which further comprises providing late acting cytokines.

27. The method of claim 26, wherein said late acting cytokines are selected from the group consisting of: granulocyte colony stimulating factor, granulocyte/macrophage colony stimulating factor, erythropoietin, FGF, EGF, NGF, VEGF, LIF, Hepatocyte growth factor and macrophage colony stimulating factor.

28. A method of treating an ischemic disease or condition in a subject in need thereof, the method comprising administering to a subject in need thereof a therapeutic amount of hematopoietic cells propagated ex-vivo by:
(a) culturing hematopoietic cells under conditions allowing for cell proliferation and,
(b) culturing said hematopoietic cells in the presence of an amount of nicotinamide for a culture period resulting in an expanded population of undifferentiated hematopoietic cells capable of enhancing perfusion in an ischemic tissue of said subject in need thereof, and
(c) administering a therapeutic amount of said expanded hematopoietic cells to said subject,
thereby treating and/or preventing said ischemic disease in said subject.

29. The method of claim 28, wherein said hematopoietic cells are from a source selected from the group consisting of: bone marrow, peripheral blood and neonatal umbilical cord blood.

30. The method of claim 28, wherein said method further comprises enriching said hematopoietic cells for hematopoietic stem cells before said culturing.

31. The method of claim 28, which further comprises enriching said expanded hematopoietic cells for hematopoietic stem cells before said administering to said subject.

32. The method of claim 28, wherein said ischemic disease or condition is a peripheral vascular disease.

33. The method of claim 28, wherein said amount of nicotinamide is about 0.1 mM to about 20 mM.

34. The method of claim 28, wherein said amount of nicotinamide is about 0.25 mM to about 15 mM.

35. The method of claim 28, wherein said amount of nicotinamide is about 0.5 mM to about 10 mM.

36. The method of claim 28, wherein said amount of nicotinamide is about 1.0 mM to about 10 mM.

37. The method of claim 28, wherein said amount of nicotinamide is about 5.0 mM.

38. The method of claim 28, wherein said culture period is about 6 days to about 6 weeks.

39. The method of claim 28, wherein said culture period is about 10 days to about 5 weeks.

40. The method of claim 28, wherein said culture period is about 2 weeks to about 4 weeks.

41. The method of claim 28, wherein said culture period is about 3 weeks.

42. The method of claim 28, wherein said culture period is about 3 weeks and said amount of nicotinamide is about 5 mM/L.

43. A method of preparing hematopoietic cells for administration to a subject suffering from an ischemic disease or condition, the method comprising:
(a) culturing hematopoietic cells under conditions allowing for cell proliferation and,
(b) culturing said hematopoietic cells in the presence of an amount of nicotinamide for a culture period resulting in an expanded population of undifferentiated hematopoietic cells capable of enhancing perfusion in an ischemic tissue of said subject in need thereof.

44. The method of claim 43, wherein said culture period is about 3 weeks and said amount of nicotinamide is about 5 mM/L.

45. The method of claim 43, wherein said ischemic disease or condition is a peripheral vascular disease.

46. An article of manufacture for treatment of peripheral vascular disease comprising a packaging material and an ex-vivo cultured hematopoietic cell population, said hematopoietic cell population propagated ex-vivo by:
(a) culturing hematopoietic cells under conditions allowing for cell proliferation and,
(b) culturing said hematopoietic cells in the presence of an amount of nicotinamide for a culture period resulting in an expanded population of undifferentiated hematopoietic cells capable of enhancing perfusion in an ischemic tissue,
and wherein said packaging material comprises a label, instructions or a package insert indicating that said hematopoietic cell population is for treating a peripheral vascular disease in a subject in need thereof.
